(19) United States
(12) Patent Application Publication
(10) Pub. No.: US 2022/0409567 A1
(43) Pub. Date: Dec. 29, 2022

(54) ADJUVANT AND COMPLEMENTARY THERAPIES FOR THE TREATMENT OF CANCER

(71) Applicant: Thomas Winston, Stillwell, KS (US)

(72) Inventor: Thomas Winston, Stillwell, KS (US)

(21) Appl. No.: 17/356,319

(22) Filed: Jun. 23, 2021

(51) Int. Cl. A61K 31/232; A61K 31/155; A61K 31/352; A61K 31/436; A61K 31/198

(52) U.S. Cl. CPC A61K 31/232; A61K 31/155; A61K 31/352; A61K 31/436; A61K 31/198

(57) ABSTRACT

Disclosed herein are pharmaceutical compositions for adjuvant and complementary therapies to treat, prevent, reduce the incidence of, or reduce the severity of cancer or one or more secondary or side effects of primary cancer treatments comprising an effective amount of one or more mTOR inhibitors and optionally an effective amount of one or more thyroid hormones. Further disclosed herein are methods of treating, preventing, reducing the incidence of, or reducing the severity of cancer and one or more secondary or side effects of primary cancer treatments comprising administering an effective amount of a pharmaceutical composition of one or more mTOR inhibitors and optionally an effective amount of one or more thyroid hormones.

### FIELD

The field of the disclosure relates generally to compositions for adjuvant and complementary therapies for the treatment of cancer. More specifically, the field of disclosure relates generally to compositions for adjuvant and complementary therapies for the treatment of cancer that include mammalian target of rapamycin (mTOR) inhibitors and optionally an effective amount of one or more thyroid hormones. More specifically, the field of disclosure relates to adjuvant and complementary therapies for the treatment of cancer when the primary method of cancer treatment includes any chemotherapy, radiation therapy, immunotherapy, a combination of any chemotherapy, radiation therapy and/or immunotherapy, or a combination of any chemotherapy, radiation therapy, and/or immunotherapy in a treatment regimen that includes any other primary cancer treatment.

### BACKGROUND

Several primary treatments for cancer can cause unwanted damage to normal cells in the body, especially chemotherapy and radiation therapy treatments. The effects of this cellular damage can cause side effects including anemia, blood clotting issues, hair loss, digestive tract issues, compromised immune system, secondary cancers and other maladies. Adjuvant and complementary therapies are often provided in cancer treatment regimens to either enhance the effectiveness of the primary cancer treatment and/or reduce these and other secondary or side effects. These adjuvant and complementary therapies may also lower the risk that the treated cancer will return, to prevent or reduce the incidence of secondary cancers forming, to preserve normal cells that would otherwise be damaged or killed by the primary treatments, and/or to preserve or rejuvenate the immune system from damage that can be caused by the primary treatments.

Some of the adverse effects associated with the occurrence of secondary or side effects as a result of chemotherapy and radiation therapy is caused by changes in the composition of cellular membranes, inflammation, and immune system dysfunction associated with the cancer or the primary cancer treatment. The composition of normal cell and organelle membranes can vary over time and in response to different pathological, nutritional and pharmacological conditions, including chemotherapy and radiation therapy, which can lead to changes in membrane structure and function. Cancer cells also display certain changes in membrane composition that may aid their ability to induce angiogenesis, establish immortality, lack response to growth suppressor signals, and undergo metastasis. And, while inflammation and immune response is necessary for development, immunity, and maintenance of tissue homeostasis and repair; inflammation and dysfunctional immune response may also be pro-tumorigenic, driving tumor initiation, growth, progression and metastasis. As a result, cancer treatment regimens have been explored to include adjuvant and complementary therapies that inhibit inflammation and/or provide for enhanced stability, survival and function of normal cells while promoting the killing and removal of cancer and other abnormal cells. However, current adjuvant and complementary therapies included in primary cancer treatment regimens are not entirely effective. New treatment approaches are needed.

### BRIEF DESCRIPTION

Disclosed herein are pharmaceutical compositions for adjuvant and complementary therapies to treat, prevent, reduce the incidence of, or reduce the severity of cancer or one or more secondary or side effects of primary cancer treatments comprising an effective amount of one or more mTOR inhibitors and optionally an effective amount of one or more thyroid hormones.

In other aspects, disclosed herein are methods of administering adjuvant and complementary therapies to treat, prevent, reduce the incidence of, or reduce the severity of cancer or one or more secondary or side effects of primary cancer treatments comprising administering an effective amount of a pharmaceutical composition of one or more mTOR inhibitors and optionally an effective amount of one or more thyroid hormones.

### DETAILED DESCRIPTION

In the following specification and the claims, reference will be made to a number of terms, which shall be defined to have the following meanings. The singular forms “a,” “an,” and “the” include plural references unless the context clearly dictates otherwise. The terms “comprising,” “including,” and “having” are intended to be inclusive and mean that there may be additional elements other than the listed elements. “Optional” or “optionally” means that the subsequently described event or a circumstance may or may not occur, and that the description includes instances where the event occurs and instances where it does not.

Approximating language, as used herein throughout the specification and claims, may be applied to modify any quantitative representation that could permissibly vary without resulting in a change in the basic function to which it is related. Accordingly, a value modified by a term or terms, such as “about,” “approximately,” and “substantially,” are not to be limited to the precise value specified. In at least some instances, the approximating language may correspond to the precision of an instrument for measuring the value. Here and throughout the specification and claims, range limitations may be combined and/or interchanged; such ranges are identified and include all the sub-ranges contained therein unless context or language indicates otherwise.

As used herein, the term “patient” refers to a warm-blooded animal such as a mammal which is the subject of a medical treatment for a medical condition that causes at least one symptom. It is understood that at least humans, dogs, cats, and horses are within the scope of the meaning of the term. In some instances, the patient is human. Generally, as used herein, the term “patient” means a human or an animal for which the compositions of the disclosure may be administered.

As used herein, the terms “treat”, “treating”, and “treatment” include inhibiting the pathological condition, disorder, or disease, e.g., arresting or reducing the development of the pathological condition, disorder, or disease or its clinical symptoms; or relieving the pathological condition, disorder, or disease, e.g., causing regression of the pathological condition, disorder, or disease or its clinical symptoms. These terms encompass also therapy and cure. Treatment may include any manner in which the symptoms of a pathological condition, disorder, or disease are ameliorated or otherwise beneficially altered.

As used herein, the term “prevent” and “preventing” includes administration of a composition which reduces the frequency of, or delays the onset of, or alleviates the symptoms of a medical condition in a subject relative to a subject which does not receive the composition.

As used herein, the term “reduce the incidence of” refers to a reduction in the number of clinical signs or symptoms of a medical condition in a subject that is administered a composition relative to a subject which does not receive the composition.

As used herein, the term “reduce the severity of” refers to a reduction in the severity of clinical signs or symptoms of a medical condition in a subject that is administered a composition relative to a subject which does not receive the composition.

As used herein, the terms “cancer” and “malignancy” refer to diseases in which abnormal cells divide without normal control and may or may not have spread to other parts of the body. Examples of different types of cancer include a carcinoma that initiates in the skin or in tissues that line or cover internal organs; a sarcoma that initiates in bone, cartilage, fat, muscle, blood vessels, or other connective or supportive tissue; a leukemia that initiates in blood-forming tissue, such as the bone marrow, and causes too many abnormal blood cells to be made; a lymphoma or multiple myeloma that initiates in the cells of the immune system; a central nervous system cancer that initiates in the tissues of the brain and spinal cord; as well as other cancers and malignancies in which abnormal cells divide without normal control.

As used herein, the term “primary treatment for cancer” refers to treatments that are designed with the primary goal of partially or completely killing or removing the abnormal cells that divide without normal control that constitute the cancer. Examples of primary treatments for cancer include surgery, chemotherapy, radiation therapy, bone marrow transplant, immunotherapy, hormone therapy, targeted drug therapy, cryoablation, and radiofrequency ablation, and combinations thereof.

As used herein, the term “adjuvant and complementary therapy” refers to therapies that are designed to treat, prevent, reduce the incidence of, or reduce the severity of cancer or one or more secondary or side effects of primary cancer treatments; to make the primary cancer treatments easier or more effective; to affect the killing of any cancer cells that remain after primary cancer treatments; to protect normal cells; to alleviate or relieve adverse symptoms of primary cancer treatments; and to be administered as part of a cancer treatment regimen before, during and/or following primary cancer treatments.

As used herein, the term “mTOR complex 1 (mTORC1)” refers to a protein complex comprising mTOR, regulatory-associated protein of mTOR (RAPTOR), mammalian lethal with SEC13 protein 8 (mLST8), proline-rich AKT substrate of 40 kDa (PRAS40) and DEP domain-containing protein 6 (DEPTOR) that has been described to function as a nutrient/energy/redox sensor; regulator of cellular growth, proliferation, and motility; and controller of protein synthesis with roles in inflammation, autophagy and cell survival.

As used herein, the term “mTOR complex 2 (mTORC2)” refers to a protein complex comprising mTOR, mLST8, DEPTOR, rapamycin-insensitive companion of mTOR (RICTOR), mammalian stress-activated protein kinase interacting protein 1 (mSIN1), and protein observed with rictor 1 and 2 (PROTOR1/2) that has been described to function as an activator of insulin receptors and insulin-like growth hormone factor 1 receptors; and regulator of cell proliferation, cell migration and cytoskeletal remodeling with roles in signaling the production of cytokines, inflammation and cell survival.

As used herein, the term “mTOR inhibitor (mTOR Inhibitor)” refers to a composition that either directly or indirectly inhibits one or more functions of mTOR, mTORC1, mTORC2 and combinations thereof. Examples of suitable mTOR inhibitors include omega-3 fatty acid derivatives, biguanide antihyperglycemic agents, flavonoids, macrolides, and other agents that effectively inhibit one or more mTOR protein complex functions.

As used herein, the term “thyroid hormone” refers to a composition that is either equivalent to, a derivative of, or affects the same functions as triiodothyronine (T3). Examples of suitable thyroid hormones include liothyronine, a T3 thyroid hormone composition.

Without being bound by theory, it is believed that the pathology of cancer involves certain changes to the structure and function of the cellular membranes and cytoskeleton that allows cells to undergo transformation, progression and metastasis. Certain similar changes are also believed to often occur in membrane and cytoskeletal structure of some surrounding normal cells. Some of the changes believed to occur involve an increase in omega-6 to omega-3 fatty acids that may interfere with the normal presentation and function of certain membrane-bound receptors including cell bound enzymes, calcium channels, sodium channels, potassium channels and other signaling proteins. As a result, the membrane bound proteins may become less responsive to stimuli including hormones, cell signaling proteins and cell signaling substances, which may in part be due to oxidative stress over time leading to changes in mTOR complex gene regulation and degradation of the omega-3 to omega-6 fatty acid ratio in cellular membranes.

Without being bound by theory, is it also believed that inflammation and improper immune response are contributing factors for predisposition to the development of cancer and promotion of tumorigenesis. It is believed that an inflammatory tumor environment exists that is promoted by inflammatory cytokines and other chemicals, some of which are released from cell membranes as a result of an imbalance of the ratio of omega-3 to omega-6 fatty acids and some of which are released from senescent cells that accumulate over time during the aging process. It is also believed that these pro-tumorigenic immune responses and inflammation also affect surrounding normal cells.

Without being bound by theory, it is also believed that mTORC1 and mTORC2 control multiple diffuse aspects of cellular metabolism, cellular integrity, cellular death, immune response and inflammation. It is believed that the mTORC1 and mTORC2 activity is enhanced and driven upwards by cytokine release including those released as a result of higher than optimal ratios of omega-6 to omega-3 fatty acids in the cell membrane and that mTORC1 and mTORC2 complex functions may be down regulated by the use of mTOR inhibitors. Examples of suitable mTOR inhibitors may include omega-3 fatty acid derivatives, biguanide antihyperglycemic agents, flavonoids, and other agents that effectively inhibit mTOR protein complexes. It is believed that omega-3 fatty acids, including icosapent ethyl, act as mTOR inhibitors at least by regulating the tumor suppressor serine/threonine kinase, LKB1, wherein LKB1 is a primary kinase for a group of other kinases that are involved in maintaining cellular energy homeostasis, growth and proliferation.

It is further believed that part of the maintenance of the cell membrane may involve maintaining an optimal ratio of omega-3 to omega-6 fatty acids, which results in an anti-inflammatory effect. It is believed that increasing the ratio of omega-3 to omega-6 fatty acids will lead to a decrease or an inhibition of cytokine production. Examples of omega-3 fatty acids include eicosapentaenoic acid (EPA) and docosahexaenoic acid (DHA), which are generally derived from diet. It is believed that EPA is superior to DHA for inhibition of inflammation and maintaining cell integrity. It is believed that omega-6 fatty acids (e.g. arachidonic acids) are precursors to the formation of cytokines. It is believed that omega-3 fatty acids may help to decrease cytokine inflammatory activity by inhibiting the formation of pro-inflammatory eicosanoid molecules like PGE2 and LTB4, by suppressing the production of pro-inflammatory cytokines like TNF-α and IL-1β, and by enhancing the formation of anti-inflammatory lipid mediators like resolvins and protectins. Accordingly, an increase of omega-3 fatty acids, in relation to omega-6, may decrease an inflammatory response caused by cytokines.

Without being bound by theory, it is believed that therapies including omega-3 fatty acids may downshift cellular signaling by decreasing cytokine formation. This may increase the maintenance of cellular adhesion and normal membrane anatomy with better sodium, potassium and calcium channel function and better response to stimuli from hormones, cell signaling proteins and other cell signaling substances such as nitric oxide. Therapies including omega-3 fatty acids may also facilitate the maintenance of membranes of mitochondria and other intracellular structures. It is further believed that control over mTOR-associated protein complexes may also be of importance in the treatment, prevention, reduction in the incidence of and reduction in the severity of cancer and secondary side effects of primary cancer treatments. It is believed that mTOR-associated protein complexes may respond to stimuli that alter cellular metabolism and growth. The mTOR-associated protein complexes may be involved in many diseases and almost all tissues of the body, including cancer. It is believed that the dysregulation of mTORC1 and mTORC2 may be an underlying cause of disease over one's lifetime. It is also believed that overactivity of these protein complexes may lead to a higher incidence and severity of cancer and secondary and side effects of primary cancer treatments.

Without being bound by theory, it is believed that adjuvant and complementary therapies for the treatment of cancer that include mTOR inhibitors may enhance the treatment, prevention, reduction of the incidence of, or reduction of the severity of cancer or one or more secondary or side effects of primary cancer treatments. It is also believed that adjuvant and complementary therapies including mTOR inhibitors may also enhance the effectiveness of primary cancer treatments, affect the killing of any cancer cells that remain after primary cancer treatments, protect normal cells, and/or alleviate or relieve adverse symptoms of primary cancer treatments. It is further believed that these adjuvant and complementary therapies may be effective if administered as part of a cancer treatment regimen before, during and/or following primary cancer treatments.

Without being bound by theory, it is believed that biguanide antihyperglycemic agents act through inhibition of the mTORC2 complex to modulate cell functions including metabolism, proliferation, migration and survival as well as reduce oxidative stress and inflammation. It is further believed that biguanide antihyperglycemic agents inhibit the mTORC2 complex by mechanisms including the reduction of the downstream effects of the AKT protein that is a component of the PI3K/AKT/mTOR pathway. The PI3K/AKT/mTOR pathway is an intracellular signaling pathway important in regulating the cell cycle and is necessary to promote growth and proliferation over differentiation of adult stem cells. However, in many cancers, this pathway is overactive, thus reducing apoptosis and allowing cell proliferation. It is further believed that biguanide antihyperglycemic agents may reduce the incidence of cancer by reducing the downstream effect of the AKT protein, which is often deregulated in cancer, in the PI3K/AKT/mTOR pathway.

Without being bound by theory, it is believed that certain flavonoids act as senolytic agents by reducing mTOR complex activity, increasing the activity of sirtuins, and increasing the activity of AMP-activated protein kinase (AMPK). These actions are believed to play a role in cellular energy homeostasis and promotion of apoptosis in senescent cells that are resistant to signaling proteins and accumulate during the aging process. It is further believed that the accumulation of senescent cells results from a weakened immune system related to aging, and these cells provide a source of chronic inflammation through the release of inflammatory chemicals and may lead to an increased risk of cancer. It is also believed that the mechanistic actions of certain flavonoids used in combination with a biguanide antihyperglycemic agent can exhibit synergistic effects for promoting apoptosis in senescent cells while promoting homeostasis in normal cells. It is further believed that when certain flavonoids are combined with certain galactomannans, the absorption of the certain flavonoids can be increased by as much as 25-fold.

Certain flavonoids, including fisetin, acting at least as a senolytic, is believed to enhance the activity of icosapent ethyl by causing cellular break down. Fisetin is also believed to affect the PI3K/AKT/mTOR pathway by downregulating the signaling pathway and enhancing lipid metabolism through its senolytic activity leading to cellular death. Fisetin is also believed to decrease inflammatory chemical production and/or release in the body, which is expected to decrease the risk of many other diseases associated with or exacerbated by inflammation.

Without being bound by theory, it is believed that administering one or more mTOR inhibitors in adjuvant and complementary therapies for the treatment of cancer may facilitate the treatment, prevention, reduction of the incidence of, or reduction of the severity of cancer or one or more secondary or side effects of primary cancer treatments. It is further believed that a composition comprised of mTOR inhibitors in adjuvant and complementary therapies may make the primary cancer treatments easier or more effective, may affect the killing of any cancer cells that remain after primary cancer treatments, may protect normal cells, and/or may alleviate or relieve adverse symptoms of primary cancer treatments. It is also believed that a composition comprised of one or more mTOR inhibitors in adjuvant and complementary therapies may be more effective if the composition is comprised of at least two or more mTOR inhibitors.

Thyroid hormones, including e.g. liothyronine (a T3 thyroid hormone), are believed to assist in controlling metabolism by utilizing oxygen and calories for conversion into energy in the mitochondria through the formation of ATP. Thyroid hormones are believed to be necessary for energy production in all organs, especially in muscle, brain, heart, and other tissues. Increased levels of thyroid hormones are believed to affect increased levels of cellular metabolism. Various tests are available to determine thyroid hormone levels, e.g. by measuring the amount of thyroid hormone levels in the blood. Thyroid hormones are believed to enhance the metabolism of fats, proteins and carbohydrates. It is further believed that treatment comprising one or more thyroid hormones in combination with fisetin may act synergistically to increase metabolism and promote the senolytic effects of fisetin.

Thyroid hormones are believed to affect nearly every cell of the body through receptors in the nucleus of the cell. Thyroid hormones bind to DNA-binding nuclear hormone receptors, cause conformational changes in the receptors, and activate transcription of the thyroid hormone sensitive genes by either initiating expression or upregulation. Also, functions of the PI3K/AKT pathway are believed to include regulation of cell adhesion, cell cycle progression, cell survival and signaling. Precursors to the thyroid hormones, referred to as T4 or thyroxine, are believed to stimulate the PI3/AKT pathway in the cytoplasm, whereas T3 does not. T3 also has a shorter half-life than T4, so T3 is recommended for adjuvant and complementary therapies for the treatment of cancer over T4.

Even though thyroid hormones are believed to increase cell survival, treatment comprising a thyroid hormone in combination with a flavonoid, such as fisetin, may act synergistically to increase metabolism and promote the senolytic effects of fisetin. It is believed that mTOR1 is upregulated in cancer cells as it facilitates metabolism, especially that of glucose, amino acids, nucleotides, fatty acids, and lipids. Inhibition of mTOR1 activity by macrolides, such as rapamycin, is believed to assist in blocking metabolism in cancer cells. Since cancer cell metabolism is generally more rapid than in normal cells, mTOR1 inhibition with rapamycin has routinely been used in the treatment of cancer. However, other than treating kidney cancer and some lymphomas, rapamycin has generally been a disappointment in the treatment of cancer in practice.

It is believed that rapamycin is primarily an mTOR1 inhibitor at lower doses and for short treatment cycles, whereas high levels and very prolonged treatment cycles can also inhibit mTOR2 by blocking mTOR2 production by the cell. Rapamycin treatment is normally administered continuously either orally or intravenously, which frequently causes side effects of insulin resistance and hyperglycemia, causes immune deficiency, and is believed to also contribute to the generation of new cancers. Also, long-term treatment with rapamycin may decrease antigen processing and inhibit T-cell proliferation leading to suppression of the immune system. Rapamycin is also believed to decrease the phosphorylation of the ribosomal s6 kinase, S6K1, which is believed to result in active decreases in protein synthesis and cell mortality.

It is believed that cancer treatment regimens that include rapamycin or a derivative of rapamycin could be much more effective and safer if rapamycin is dosed at low-levels either intermittently or in conjunction with other mTOR inhibitors and/or additional medications that decrease or down regulate the PI3K-AKT pathway. It is believed that these treatment regimens will alter metabolism and inhibit the ability of cancer cells to replicate. It is further believed that using a biguanide antihyperglycemic agent, such as metformin, in these treatment regimens will allow for down regulation of both mTOR1 and mTOR2 safely without causing significant side effects of high-dose rapamycin. In addition to acting as an inhibitor of mTOR2, metformin also decreases glycolysis and is effective in controlling blood glucose levels. The addition of a flavonoid, such as fisetin, is believed to provide the added benefit of promoting apoptosis or cell death effectuated at least partly through its inhibition of the mTOR pathway. The effects of fisetin may be further improved with the addition of a T3 thyroid hormone.

It is believed that synergy of activity for inhibition of the PI3K-AKT pathway can be achieved with the combination of rapamycin, metformin, and fisetin while providing a low risk of side effects. The treatment regimens may further benefit from the addition of an omega-3 fatty acid derivative, which is believed to downregulate mTOR2, decrease cytokine formation, strengthen cell membranes and structures, and decrease phosphorylation of phosphatides. Additionally, the addition of a T3 thyroid hormone is believed to enhance the effectiveness of the therapy regimen. These combination therapies are believed to have minimal side effects, may be administered continuously over long periods of time, and result in an effective decrease in PI3K-AKT activity, cell proliferation, survival, and growth of cancer cells. These combination therapies are believed to be effective as primary cancer treatments as well as adjuvant and complementary treatments of cancer including long-term administration to affect a cancer-free cure or to maintain a sustainable and survivable low-level presence of cancer. As adjuvant and complementary treatments, these therapies can be prescribed with other drug therapies, alongside chemotherapy, radiation therapy, immunotherapy or any other primary cancer treatments or combinations of cancer therapies.

In various embodiments, the compositions of the disclosure include compositions for adjuvant and complementary therapies for the treatment of cancer. In various embodiments, the compositions of the disclosure include an effective amount of one or more mTOR inhibitors and optionally an effective amount of one or more thyroid hormones. In various embodiments, suitable mTOR inhibitors may include omega-3 fatty acid derivatives, biguanide antihyperglycemic agents, flavonoids, macrolides and other agents that effectively inhibit mTOR protein complexes. In various embodiments, suitable thyroid hormones may include a T3 hormone, such as liothyronine. Preferentially, at least one of the components of the composition will decrease pro-tumorigenic inflammation associated with cancer and primary cancer treatments.

Preferentially, at least one of the components of the composition will decrease the rate of normal cell death or will increase the life span of normal cells including those involved in immune response systems.

Preferentially, at least one of the components of the composition will enhance cellular membrane integrity and function and/or induce apoptosis in cancer and/or senescent cells.

In various embodiments, the compositions may include an effective amount of an omega-3 fatty acid derivative. Suitable omega-3 fatty acid derivatives may include icosapent ethyl. In various embodiments, the compositions of the disclosure may include an effective amount of at least about 0.5 g of icosapent ethyl, or between about 0.5 g to about 10.0 g, or 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 g, or any range between any two of these amounts including about 0.5 g to about 10.0 g, or about 1.0 g to about 7.0 g, or about 2.0 g to about 8.0 g, or about 2.0 g to about 4.0 g, or about 4.0 g to about 8.0 g. In some preferred forms, the amount of icosapent ethyl is sufficient to maintain an optimum level of icosapent ethyl in the blood of a subject receiving an administration of the composition.

In various embodiments, the compositions of the disclosure may include an effective amount of a biguanide antihyperglycemic agent. Suitable biguanide antihyperglycemic agents include metformin. In various embodiments, the compositions may include an effective amount of at least about 50 mg of biguanide antihyperglycemic agent, or between about 50 mg to about 4000 mg, or 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1100, 1125, 1150, 1175, 1200, 1225, 1250, 1275, 1300, 1325, 1350, 1375, 1400, 1425, 1450, 1475, 1500, 1525, 1550, 1575, 1600, 1625, 1650, 1675, 1700, 1725, 1750, 1775, 1800, 1825, 1850, 1875, 1900, 1925, 1950, 1975, 2000, 2025, 2050, 2075, 2100, 2125, 2150, 2175, 2200, 2225, 2250, 2275, 2300, 2325, 2350, 2375, 2400, 2425, 2450, 2475, 2500, 2525, 2550, 2575, 2600, 2625, 2650, 2675, 2700, 2725, 2750, 2775, 2800, 2825, 2850, 2875, 2900, 2925, 2950, 2975, 3000, 3025, 3050, 3075, 3100, 3125, 3150, 3175, 3200, 3225, 3250, 3275, 3300, 3325, 3350, 3375, 3400, 3425, 3450, 3475, 3500, 3525, 3550, 3575, 3600, 3625, 3650, 3675, 3700, 3725, 3750, 3775, 3800, 3825, 3850, 3875, 3900, 3925, 3950, 3975, or 4000 mg or any range between any two of these amounts including about 250 mg to about 4000 mg, about 250 mg to about 500 mg, about 250 mg to about 750 mg, 250 mg to about 1000 mg, about 250 mg to about 1250 mg, about 250 mg to about 1500 mg, or about 500 mg to about 3000 mg, or about 500 mg to about 2000 mg, or about 1000 mg to about 4000 mg.

In various embodiments, the compositions may include an effective amount of a flavonoid. Suitable flavonoid agents include fisetin and fisetin derivatives. In various embodiments, the compositions may include an effective amount of at least about 10 mg/kg of patient body weight of a flavonoid, or between about 10 mg/kg to about 100 mg/kg of patient body weight, or 10, 15, 20, 25, 30, 35, 40, 45, 50, 55, 60, 65, 70, 75, 80, 85, 90, 95, or 100 mg/kg of patient body weight or any range between any two of these amounts including about 10 mg/kg to about 20 mg/kg, about 15 mg/kg to about 25 mg/kg, about 20 mg/kg to about 30 mg/kg, about 25 mg/kg to about 50 mg/kg, or about 20 mg/kg to about 100 mg/kg of a flavonoid. In some preferred forms, the amount of fisetin is sufficient to maintain an optimum level of fisetin in the blood of a subject receiving an administration of the composition, such optimum level may be achieved by combining the fisetin with a galactomannan to enhance the absorption of the flavonoid. In some preferred forms, the amount of fisetin is as close to the dose of fisetin that is tolerated by the patient. In some preferred forms, the amount of fisetin in the compositions is increased on a daily basis to an optimum level or optimum high dose level of fisetin.

In various embodiments, the compositions may include an effective amount of a flavonoid. Suitable flavonoid agents include fisetin and fisetin derivatives. In various embodiments, the compositions may include an effective amount of at least about 50 mg of a flavonoid, or between about 50 mg to about 750 mg, or 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, or 750 mg or any range between any two of these amounts including about 50 mg to about 500 mg, about 100 mg to about 750 mg, about 250 mg to about 500 mg, about 250 mg to about 750 mg, about 500 mg to about 750 mg, or about 100 mg to about 500 mg of a flavonoid. In some preferred forms, the amount of fisetin is sufficient to maintain an optimum level of fisetin in the blood of a subject receiving an administration of the composition; such optimum level may be achieved by combining the fisetin with a galactomannan to enhance the absorption of the flavonoid. In some preferred forms, the amount of fisetin is as close to the dose of fisetin that is tolerated by the patient. In some preferred forms, the amount of fisetin in the compositions is increased on a daily basis to an optimum level or optimum high dose level of fisetin.

In various embodiments, the compositions may include an effective amount of a macrolide. Suitable macrolides include rapamycin. In various embodiments, the compositions may include an effective amount of a macrolide of at least about 0.1 mg of a macrolide, or between about 0.1 mg to about 10 mg, or 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 mg or any range between any two of these amounts including about 2.0 mg to about 6.0 mg, about 1.0 mg to about 10.0 mg, about 2.0 mg to about 4.0 mg, about 2.5 mg to about 5.0 mg, about 2.5 mg to about 7.5 mg, or about 1.0 mg to about 5.0 mg of a macrolide. In some preferred forms, the amount of rapamycin is administered as a loading dose followed by a lower daily dose. In some preferred forms, the amount of rapamycin is sufficient to maintain an optimum level of rapamycin in the blood of a subject receiving an administration of the composition; such optimum level may be determined as a preferred optimum trough level as measured in nanograms per ml of blood. In some preferred forms, the administration of rapamycin is provided intermittently at low levels.

In various embodiments, the compositions may include an effective amount of a thyroid hormone. Suitable thyroid hormones include the T3 liothyronine. In various embodiments, the compositions of the disclosure may include an effective amount of at least about 1 μg of liothyronine, or between about 1 μg to about 250 μg, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250 μg, or any range between any two of these amounts including about 5 μg to about 150 μg, or about 10 μg to about 100 μg, or about 10 μg to about 25 μg, or about 25 μg to about 150 μg, or about 25 μg to about 250 μg. In some preferred embodiments, thyroid hormones are not administered on a daily basis and administration does not exceed three days per week. In some preferred methods, the administration of thyroid hormones is dependent upon the clinical response and tolerance of the patient and may continue long-term including many years. In some preferred methods, the amount of liothyronine administered is sufficient to maintain an optimum level of liothyronine in the blood of a subject receiving an administration of the composition. In various embodiments, the compositions may include a combination of an effective amount of one or more thyroid hormones with either an effective amount of a flavonoid or a high dose of a flavonoid, wherein only some of the compositions that include an effective amount of a flavonoid also include an effective amount of one or more thyroid hormones. For example, an effective amount of one or more thyroid hormones may be included only in one of two weekly compositions administered that includes an effective amount of a flavonoid or a high dose of a flavonoid, which composition could be either the first or second weekly composition administered that includes a flavonoid or high dose flavonoid.

In various embodiments, the compositions may include an effective amount of a combination of thyroid hormones. Suitable thyroid hormones for combination include the T3 liothyronine. In various embodiments, the compositions may include an effective amount of a combination of one or more thyroid hormones with an effective amount of a flavonoid. In various embodiments, the compositions may include an effective amount of one or more thyroid hormones and an effective amount of a high dose of a flavonoid, which is associated with a senolytic effect. In various embodiments, the compositions may include an effective amount of one or more thyroid hormones with an effective of either a flavonoid or a high dose flavonoid only in certain daily administrations of compositions including a flavonoid.

In various embodiments, the compositions may include an effective amount of a combination of one or more mTOR inhibitors with an effective amount of one or more thyroid hormones. In various embodiments, the compositions may include an effective amount of one or more mTOR inhibitors with an effective amount of one or more thyroid hormones and an effective amount of a flavonoid. In various embodiments, the compositions may include an effective amount of one or more mTOR inhibitors with an effective amount of one or more thyroid hormones and an effective amount of a high dose of a flavonoid, which is associated with a senolytic effect. In various embodiments, the compositions may include an effective loading dose of one or more mTOR inhibitors followed by an effective amount of a daily dose of one or more mTOR inhibitors. In various embodiments, the compositions may include an effective loading dose of one or more mTOR inhibitors followed by an effective amount of a weekly dose of one or more mTOR inhibitors.

In various embodiments, the compositions of the disclosure may further contain additional pharmaceutically acceptable carriers. The pharmaceutical compositions may, for example, be in a form suitable for oral administration as a tablet, capsule, pill, powder, sustained release formulation, solution or suspension, in a form suitable for parenteral injection as a sterile solution, suspension, or in a form of an emulsion for topical administration as an ointment or cream or for rectal administration as a suppository. The pharmaceutical compositions may be in unit dosage forms suitable for single administration of precise dosages. The pharmaceutical compositions may include conventional pharmaceutical carriers or excipients. In addition, the compositions may include other medicinal or pharmaceutical agents, carriers, adjuvants, etc. In various embodiments, the compositions may be administered to a patient through any suitable route of administration effective in delivering an amount of active agent or active agents to a patient. Suitable routes of administration include oral, parenteral, enteral, and rectal or the like.

In some forms, the composition will comprise each of the ingredients in a single administration form, such as a pill, tablet, capsule, oral solution, injection solution, infusion solution, or any of the forms described herein. In other forms, the composition will comprise a kit comprising each of the individual ingredients, together with instructions for administering each ingredient. In some forms of the kit, certain ingredients will already be combined such that two, three, or more of the components or ingredients of the composition are in a single administration form as described herein.

Various embodiments of the disclosure further relate to methods of treating cancer that include adjuvant and complementary therapies comprising the administration of a composition of an effective amount of one or more mTOR inhibitors and optionally an effective amount of one or more thyroid hormones in addition to the primary cancer treatment in a cancer treatment regimen. In various embodiments, suitable mTOR inhibitors may include omega-3 fatty acid derivatives, biguanide antihyperglycemic agents, flavonoids, macrolides and other agents that effectively inhibit mTOR protein complexes. In various embodiments, suitable thyroid hormones may include a T3 hormone, such as liothyronine.

In various embodiments, the methods may include administering an effective amount of an omega-3 fatty acid derivative. Suitable omega-3 fatty acid derivatives may include icosapent ethyl. In various embodiments, the methods may include administering an effective amount of at least about 0.5 g of icosapent ethyl, or between about 0.5 g to about 10.0 g, or 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 g, or any range between any two of these amounts including about 0.5 g to about 10.0 g, or about 1.0 g to about 7.0 g, or about 2.0 g to about 8.0 g, or about 2.0 g to about 4.0 g, or about 4.0 g to about 8.0 g once, twice, or three or more times daily. In some preferred forms, the amount of icosapent ethyl is sufficient to maintain an optimum level of icosapent ethyl in the blood of a subject receiving an administration of the composition on a daily basis.

In various embodiments, the methods may include administering an effective amount of a biguanide antihyperglycemic agent. Suitable biguanide antihyperglycemic agents include metformin. In various embodiments, the methods may include administering an effective amount of at least about 50 mg of biguanide antihyperglycemic agent, or between about 50 mg to about 4000 mg, or 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1100, 1125, 1150, 1175, 1200, 1225, 1250, 1275, 1300, 1325, 1350, 1375, 1400, 1425, 1450, 1475, 1500, 1525, 1550, 1575, 1600, 1625, 1650, 1675, 1700, 1725, 1750, 1775, 1800, 1825, 1850, 1875, 1900, 1925, 1950, 1975, 2000, 2025, 2050, 2075, 2100, 2125, 2150, 2175, 2200, 2225, 2250, 2275, 2300, 2325, 2350, 2375, 2400, 2425, 2450, 2475, 2500, 2525, 2550, 2575, 2600, 2625, 2650, 2675, 2700, 2725, 2750, 2775, 2800, 2825, 2850, 2875, 2900, 2925, 2950, 2975, 3000, 3025, 3050, 3075, 3100, 3125, 3150, 3175, 3200, 3225, 3250, 3275, 3300, 3325, 3350, 3375, 3400, 3425, 3450, 3475, 3500, 3525, 3550, 3575, 3600, 3625, 3650, 3675, 3700, 3725, 3750, 3775, 3800, 3825, 3850, 3875, 3900, 3925, 3950, 3975, or 4000 mg or any range between any two of these amounts including about 250 mg to about 4000 mg, about 250 mg to about 500 mg, about 250 mg to about 750 mg, 250 mg to about 1000 mg, about 250 mg to about 1250 mg, about 250 mg to about 1500 mg, or between about 500 mg to about 3000 mg, or about 500 mg to about 2000 mg, or about 1000 mg to about 4000 mg once, twice, or three or more times daily.

In various embodiments, the method may include administering an effective amount of a flavonoid. Suitable flavonoid agents include fisetin and fisetin derivatives. In various embodiments, the methods may include administering an effective amount of at least about 10 mg/kg of patient body weight of a flavonoid, or between about 10 mg/kg to about 100 mg/kg of patient body weight, or 10, 15, 20, 25, 30, 35, 40, 45, 50, 55, 60, 65, 70, 75, 80, 85, 90, 95, or 100 mg/kg of patient body weight or any range between any two of these amounts including about 10 mg/kg to about 20 mg/kg, about 15 mg/kg to about 25 mg/kg, about 20 mg/kg to about 30 mg/kg, about 25 mg/kg to about 50 mg/kg, or about 20 mg/kg to about 100 mg/kg once, twice, or three or more times daily, weekly, monthly, trimonthly or intermittently with periods between administration when no flavonoid is administered. In some preferred methods, the flavonoid may be administered only one or two days per week, or only one or two days every two weeks, or only one or two days every three weeks or only one or two days per month, bimonthly or trimonthly. In some preferred methods, the flavonoid may be administered each day at the highest specified dose that the patient can tolerate. In various embodiments, the flavonoid or high dose of the flavonoid may include long-term administration, possibly for the life of the patient. In some preferred methods, the amount of fisetin is sufficient to maintain an optimum level of fisetin in the blood of a subject receiving an administration of the composition; such optimum level may be achieved by combining the fisetin with a galactomannan to enhance the absorption of the flavonoid. In various embodiments, a higher dose of fisetin may be associated with a senolytic effect. In various embodiments, a lower dose of fisetin may be associated with an antioxidant effect. In some preferred forms, the amount of fisetin is as close to the dose of fisetin that is tolerated by the patient. In some preferred forms, the amount of fisetin in the compositions is increased on a daily basis to an optimum level or optimum high dose level of fisetin.

In various embodiments, the methods may include administering an effective amount of a flavonoid that is administered on a daily basis. Suitable flavonoid agents include fisetin and fisetin derivatives. In various embodiments, the methods may include administering an effective amount of at least about 50 mg of a flavonoid, or between about 50 mg to about 750 mg, or 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, or 750 mg or any range between any two of these amounts including about 50 mg to about 500 mg, about 100 mg to about 750 mg, about 250 mg to about 500 mg, about 250 mg to about 750 mg, about 500 mg to about 750 mg, or about 100 mg to about 500 mg once, twice, or three or more times daily. In some preferred methods, the flavonoid may be administered only one or two days per week, or only one or two days every two weeks, or only one or two days every three weeks or only one or two days per month, bimonthly or trimonthly. In some preferred methods, the flavonoid may be administered each day at the highest specified dose that the patient can tolerate. In various embodiments, the flavonoid or high dose of the flavonoid may include long-term administration, possibly for the life of the patient. In some preferred methods, the amount of fisetin is sufficient to maintain an optimum level of fisetin in the blood of a subject receiving an administration of the composition; such optimum level may be achieved by combining the fisetin with a galactomannan to enhance the absorption of the flavonoid. In various embodiments, a higher dose of fisetin may be associated with a senolytic effect. In various embodiments, a lower dose of fisetin may be associated with an antioxidant effect.

In various embodiments, the methods may include administering an effective amount of a macrolide. Suitable macrolides include rapamycin. In various embodiments, the methods may include administering an effective amount of a macrolide of at least about 0.1 mg of a macrolide, or between about 0.1 mg to about 10 mg, or 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 mg or any range between any two of these amounts including about 2.0 mg to about 6.0 mg, about 1.0 mg to about 10.0 mg, about 2.0 mg to about 4.0 mg, about 2.5 mg to about 5.0 mg, about 2.5 mg to about 7.5 mg, or about 1.0 mg to about 5.0 mg of a macrolide. In some preferred forms, the amount of rapamycin is administered as a loading dose followed by a lower daily dose. In some preferred forms, the amount of rapamycin is sufficient to maintain an optimum level of rapamycin in the blood of a subject receiving an administration of the composition; such optimum level may be determined as a preferred optimum trough level as measured in nanograms per ml of blood. In some preferred forms, the administration of rapamycin is provided intermittently at low levels.

In various embodiments, the methods may include administering an effective amount of an omega-3 fatty acid derivative in a dosing regimen with an effective amount of a biguanide antihyperglycemic agent.

In various embodiments, the methods may include administering an effective amount of an omega-3 fatty acid derivative in a dosing regimen with an effective amount of a flavonoid.

In various embodiments, the methods may include administering an effective amount of an omega-3 fatty acid derivative in a dosing regimen with an effective amount of a biguanide antihyperglycemic agent and an effective amount of a flavonoid.

In various embodiments, the methods may include administering an effective amount of a biguanide antihyperglycemic agent in a dosing regimen with an effective amount of a flavonoid.

In various embodiments, the methods may include administering the effective amount of the compositions before, during and/or following primary cancer treatments.

In various embodiments, the methods may include administering an effective amount of a thyroid hormone. Suitable thyroid hormones include the T3 liothyronine. In various embodiments, the methods of the disclosure may include administering an effective amount of at least about 1 μg of liothyronine, or between about 1 μg to about 250 μg, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250 μg, or any range between any two of these amounts including about 5 μg to about 150 μg, or about 10 μg to about 100 μg, or about 10 μg to about 25 μg, or about 25 μg to about 150 μg, or about 25 μg to about 250 μg either weekly, bimonthly, or monthly. In some preferred embodiments, thyroid hormones are not administered on a daily basis and administration does not exceed three days per week. In some preferred methods, the administration of thyroid hormones is dependent upon the clinical response and tolerance of the patient and may continue long-term including many years. In some preferred methods, the amount of liothyronine administered is sufficient to maintain an optimum level of liothyronine in the blood of a subject receiving an administration of the composition.

In various embodiments, the methods may include diagnosing thyroid functions in each patient prior to administration of an effective amount of a thyroid hormone. In various embodiments, the methods for patients requiring thyroid hormone replacement in order to establish normal thyroid functions may preferentially be administered a T3 thyroid hormone. In various embodiments, patients with normal thyroid functions may be administered a low dose of a T3 thyroid hormone (e.g. 5 to 10 μg of liothyronine) combined with a high dose of a flavonoid. In various embodiments, the methods may include administering a combination of a low dose of a T3 thyroid hormone and a high dose of a flavonoid that effectively elicits a synergistic effect of increasing metabolism and promoting cellular senescence.

In various embodiments, the methods may include administering an effective amount of a combination of thyroid hormones. Suitable thyroid hormones that may be included in the combination include the T3 liothyronine. In various embodiments, the methods may include administering an effective amount of a combination of one or more thyroid hormones with an effective amount of a flavonoid. In various embodiments, the methods may include administering an effective amount of one or more thyroid hormones and an effective amount of a high dose of a flavonoid, which is associated with a senolytic effect. In various embodiments, the methods may include administering a combination of an effective amount of one or more thyroid hormones with either an effective amount of a flavonoid or a high dose of a flavonoid, wherein only some of the compositions that include an effective amount of a flavonoid or high does flavonoid also include an effective amount of one or more thyroid hormones. For example, an effective amount of one or more thyroid hormones may be included only in one of two weekly compositions administered that includes an effective amount of a flavonoid or a high dose of a flavonoid, which composition could be either the first or second weekly composition administered that includes a flavonoid or high dose flavonoid.

In various embodiments, the methods may include administering an effective amount of a combination of one or more mTOR inhibitors with an effective amount of one or more thyroid hormones. In various embodiments, the methods may include administering an effective amount of one or more mTOR inhibitors with an effective amount of one or more thyroid hormones and an effective amount of a flavonoid. In various embodiments, the methods may include administering an effective amount of one or more mTOR inhibitors with an effective amount of one or more thyroid hormones and an effective amount of a high dose of a flavonoid, which is associated with a senolytic effect unless the patient is hypothyroid on the days of the week the thyroid hormone is administered. In various embodiments, the methods may include administering a combination of an effective amount of one or more mTOR inhibitors with an effective amount of one or more thyroid hormones and either an effective amount of a flavonoid or a high dose of a flavonoid, wherein only some of the compositions that include an effective amount of a flavonoid also include an effective amount of one or more thyroid hormones. For example, an effective amount of one or more thyroid hormones may be included only in one of two weekly compositions administered that includes an effective amount of a flavonoid or a high dose of a flavonoid, which composition could be either the first or second weekly composition administered that includes a flavonoid or high dose flavonoid. In various embodiments, the one or more thyroid hormones should be administered for short durations (for example, two days a week, bimonthly, or monthly) during any periods of a dosing regimen that include a high dose of a flavonoid.

In various embodiments, the methods may include administering an effective loading dose of one or more mTOR inhibitors followed by an effective amount of a daily dose of one or more mTOR inhibitors. In various embodiments, the methods may include administering an effective loading dose of one or more mTOR inhibitors followed by an effective amount of a weekly dose of one or more mTOR inhibitors.

In various embodiments, the methods may include administering an effective loading dose of one or more mTOR inhibitors followed by an effective amount of a weekly dose of one or more mTOR inhibitors.

In some preferred forms, the methods include administering a macrolide, such as rapamycin; in combination with a biguanide antihyperglycemic agent, such as metformin; in combination with an omega-3 fatty acid derivative, such as icosapent ethyl; in combination with a flavonoid, such as fisetin; and in combination with a T3 thyroid hormone, such as liothyronine. In some preferred forms, the methods include administering a macrolide, such as rapamycin, dosed weekly to achieve blood levels below specified levels measured at specified times following administration; in combination with a biguanide antihyperglycemic agent, such as metformin, dosed twice daily; in combination with an omega-3 fatty acid derivative, such as icosapent ethyl, dosed twice daily; in combination with a flavonoid, such as fisetin, dosed daily; in combination with a T3 thyroid hormone, such as liothyronine, dosed either for two days in a row per week or dosed on separate days for no more than 3 days per week. In some preferred forms, the methods include administering a macrolide, such as rapamycin, dosed weekly to achieve blood levels below about 12 nanograms per ml of blood measured at about 60 hours plus or minus 3 hours following administration; in combination with a biguanide antihyperglycemic agent, such as metformin, dosed twice daily at about 500 mg to about 2000 mg; in combination with an omega-3 fatty acid derivative, such as icosapent ethyl, dosed twice daily at about 2.0 g to about 4.0 g; in combination with a flavonoid, such as fisetin, dosed daily at about 20.0 mg/kg patient body weight (which dose may be achieved by starting at a daily dose of about 200 mg and stepping up to the daily dose of about 20.0 mg/kg); in combination with a T3 thyroid hormone, such as liothyronine, dosed either for two days in a row per week or dosed on separate days for no more than 3 days per week at about 5.0 μg to about 10.0 μg. In some preferred forms, the methods include administering a macrolide, such as rapamycin, dosed to achieve and maintain blood levels of about 12 to about 20 nanograms per ml of blood for a short or prolonged period followed by the administration of additional agents to the treatment regimen, such additional agents comprising a biguanide antihyperglycemic agent, such as metformin, an omega-3 fatty acid derivative, such as icosapent ethyl, a flavonoid, such as fisetin, and a T3 thyroid hormone, such as liothyronine.

In various embodiments, the methods may include administering the effective amount of the compositions of the disclosure that may further contain additional pharmaceutically acceptable carriers, excipients, other medicinal or pharmaceutical agents, carriers, adjuvants, etc.

In various embodiments, the methods may include administering the effective amount of the compositions to a patient through any suitable route of administration effective in delivering an amount of active agent or active agents to a patient. Suitable routes of administration include oral, intravascular, intramuscular, subcutaneous, parenteral, enteral, and rectal or the like.

In various embodiments, the methods may include administering the effective amount of the compositions comprised of each of the ingredient in a single administration form, such as a pill, tablet, capsule, oral solution, injection solution, infusion solution, or any of the forms described herein. In various embodiments, the methods may include administering the effective amount of the compositions from a kit comprising each of the individual ingredients, together with instructions for administering each ingredient. In some forms of the kit, certain ingredients will already be combined such that one, two, three, four, or more of the components or ingredients of the composition are in a single administration form as described herein.

This written description uses examples to disclose the subject matter herein, including the best mode, and also to enable any person skilled in the art to practice the subject matter disclosed herein, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A pharmaceutical composition for adjuvant and complementary therapy to treat, prevent, reduce the incidence of, or reduce the severity of cancer or one or more secondary effects of primary cancer treatments comprising an effective amount of one or more mTOR inhibitors.

2. The pharmaceutical composition of claim 1, wherein the one or more mTOR inhibitors is selected from the group consisting of: an omega-3 fatty acid derivative, a biguanide antihyperglycemic agent, a flavonoid, a macrolide, and combinations thereof.

3. The pharmaceutical composition of claim 2, wherein the omega-3 fatty acid derivative is icosapent ethyl.

4. The pharmaceutical composition of claim 2, wherein the biguanide antihyperglycemic agent is metformin.

5. The pharmaceutical composition of claim 2, wherein the flavonoid is selected from the group consisting of fisetin and fisetin derivatives.

6. The pharmaceutical composition of claim 2, wherein the macrolide is rapamycin.

7. The pharmaceutical composition of claim 2, wherein the pharmaceutical composition further comprises an effective amount of one or more thyroid hormones.

8. The pharmaceutical composition of claim 7, wherein the one or more thyroid hormones comprises liothyronine.

9. The pharmaceutical composition of claim 8, wherein the flavonoids are selected from the group consisting of fisetin and fisetin derivatives.

10. The pharmaceutical composition of claim 7, wherein the omega-3 fatty acid derivative is icosapent ethyl, wherein the biguanide antihyperglycemic agent is metformin, wherein the flavonoid is fisetin, wherein the macrolide is rapamycin, and wherein the one or more thyroid hormones comprises liothyronine.

11. A method of treating, preventing, reducing the incidence of, or reducing the severity of cancer or one or more secondary effects of primary cancer treatments comprising administering an effective amount of a pharmaceutical composition of one or more mTOR inhibitors.

12. The method of claim 11, wherein the one or more mTOR inhibitors is selected from the group consisting of: an omega-3 fatty acid derivative, a biguanide antihyperglycemic agent, a flavonoid, a macrolide and combinations thereof.

13. The method of claim 12, wherein the omega-3 fatty acid derivative is icosapent ethyl.

14. The method of claim 12, wherein the biguanide antihyperglycemic agent is metformin.

15. The method of claim 12, wherein the flavonoid is selected from the group consisting of fisetin and fisetin derivatives.

16. The method of claim 12, wherein the macrolide is rapamycin.

17. The method of claim 12, wherein the pharmaceutical composition further comprises an effective amount of one or more thyroid hormones.

18. The method of claim 17, wherein the one or more thyroid hormones comprises liothyronine.

19. The method of claim 18, wherein the flavonoid is selected from the group consisting of fisetin and fisetin derivatives.

20. The method of claim 17, wherein the omega-3 fatty acid derivative is icosapent ethyl, wherein the biguanide antihyperglycemic agent is metformin, wherein the flavonoid is fisetin, wherein the macrolide is rapamycin, and wherein the one or more thyroid hormones comprises liothyronine.

21. The method of claim 17, wherein the macrolide is administered prior to the administration of any combined one or more mTOR inhibitors and the one or more thyroid hormones.
